(19) United States
(12) Patent Application Publication
(10) Pub. No.: US 2023/0142197 A1
(43) Pub. Date: May 11, 2023

(54) CATION EXCHANGE FOR SPERM-ASSOCIATED DNA PURIFICATION

(71) Applicant: Promega Corporation, Madison, WI (US)

(72) Inventors: Nicholas A. Courtney, Madison, WI (US); Robert Stewart McLaren, Oregon, WI (US); Zhiyang Zeng, Madison, WI (US); Jian Cao, Madison, WI (US); Wenhui Zhou, Madison, WI (US)

(21) Appl. No.: 18/054,841

(22) Filed: Nov. 11, 2022

Related U.S. Application Data
(60) Provisional application No. 63/278,341, filed on Nov. 11, 2021.

(51) Int. Cl. C12N 15/10; C12Q 1/6888; C12Q 1/6806

(52) U.S. Cl. CPC C12N 15/101; C12Q 1/6888; C12Q 1/6806; C12Q 2600/156

(57) ABSTRACT

Provided herein are methods, reagents, and kits for the isolation of sperm cells from other cell types and the purification of sperm-associated DNA from a complex sample. In particular, methods, reagents, and kits are provided for the separation of intact sperm cells from epithelial cells via binding of sperm cells to a cation exchange resin, and the extraction of sperm-associated DNA therefrom.

### CROSS-REFERENCE TO RELATED APPLICATIONS

This application claims the benefit of U.S. Provisional Patent Application No. 63/278,341, filed on Nov. 11, 2021, which is incorporated by reference herein.

### FIELD

Provided herein are methods, reagents, and kits for the isolation of sperm cells from other cell types and the purification of sperm-associated DNA from a complex sample. In particular, methods, reagents, and kits are provided for the separation of intact sperm cells from epithelial cells via binding of sperm cells to a cationic exchange material, and the extraction of sperm-associated DNA therefrom.

### BACKGROUND

In the field of forensic DNA analysis, there is a major need to be able to efficiently separate sperm-associated DNA from materials collected with a sexual assault kit, including the victim's DNA. Clean separation of sperm-associated DNA is essential to obtaining short tandem repeat (STR) profiles of potential suspects. The most common process for separating sperm-associated DNA, termed Differential Extraction (DE), is a labor-intensive protocol that requires a high degree of technical prowess and is incompatible with most automation techniques. In part due to the difficulties of the current protocol, there is a significant nationwide back log of sexual assault kits that are awaiting testing.

Thus, there remains a need for easy to use, cost-effective methods and kits for isolating sperm-associated DNA.

### SUMMARY

Provided herein are methods, reagents, and kits for the isolation of sperm cells from other cell types and the purification of sperm-associated DNA from a complex sample. In particular, methods, reagents, and kits are provided for the separation of intact sperm cells from epithelial cells via binding of sperm cells to a cation exchange material, and the extraction of sperm-associated DNA therefrom.

Specifically, the disclosure provides a method comprising: (a) contacting a sample comprising sperm cells with a digestion agent capable of efficiently lysing epithelial cells but not sperm cells; (b) separating a sperm-cell-enriched fraction of the sample from a sperm-cell-depleted fraction of the sample; (c) contacting the sperm-cell-enriched fraction with a cationic exchange resin; (e) washing the cationic exchange resin with a wash buffer; and (f) eluting sperm-associated DNA from the cationic exchange resin. In other embodiments, the disclosure provides a method comprising: (a) contacting a sample comprising sperm cells with a digestion agent capable of efficiently lysing epithelial cells but not sperm cells; (b) contacting the sample with a cationic exchange resin; (c) washing the cationic exchange resin with a wash buffer; and (d) eluting sperm-associated DNA from the cationic exchange resin

The disclosure further provides a kit comprising two or more of: (a) a digestion agent capable of efficiently lysing epithelial cells but not sperm cells; (b) a cationic exchange resin; (c) a resuspension buffer; (d) a wash buffer; and (e) an elution buffer comprising a digestion agent and a reducing agent.

### BRIEF DESCRIPTION OF THE DRAWINGS

FIG. 1 is a graph showing yield data for semen samples. Data was obtained from a single experiment; each dot represents a sample (n=2 per condition) while the bars represent the mean for each condition. Liquid semen (1 μl) was used as the input material for each sample. Data was quantified using Promega's POWERQUANT™ system.

FIG. 2 is a graph illustrating that multiple cation exchange particles can purify sperm-associated DNA from semen samples. Data was obtained from a single experiment; each dot represents a sample (n=2 per condition) while the bars represent the mean for each condition. Liquid semen (1 μl) was used as the input material for each sample, and 5 mg of the indicated cationic exchange resins were added to the MAXWELL® cartridge. Data was quantified using Promega's POWERQUANT™ system.

FIG. 3 is a graph showing yield data for purified DNA samples. Data was obtained from a single experiment; each dot represents a sample (n=2 per condition) while the bars represent the mean for each condition. DNA (50 ng 2800M Control DNA per sample; Promega Catalogue #: DD7101) was used as the input material for each sample, and the input control condition was created by diluting 50 ng of 2800M DNA into the volume of the elution buffer.

FIG. 4 is a graph showing data examining the yield of sperm-associated DNA as a function of resin mass. Data was obtained from a single experiment; each dot represents a sample (n=2 per condition). Liquid semen (1 μl) was used as the input material for each sample, and the differing masses of resin was added to the MAXWELL® cartridge as indicated. Data was quantified using Promega's POWERQUANT™ system.

FIG. 5 is a graph showing data comparing functionalized and parent resins. Data was obtained from a single experiment; each dot represents a sample (n=2 per condition) while the bars represent the mean for each condition. Liquid semen (1 μl) was used as the input material for each sample. Data was quantified using Promega's POWERQUANT™ system.

FIG. 6 is a graph showing data from control inputs. Data was obtained from a single experiment; each dot represents a sample (n=3 per condition) while the bars represent the mean for each condition. The input materials are as noted for each condition. Data was quantified using Promega's POWERQUANT™ system.

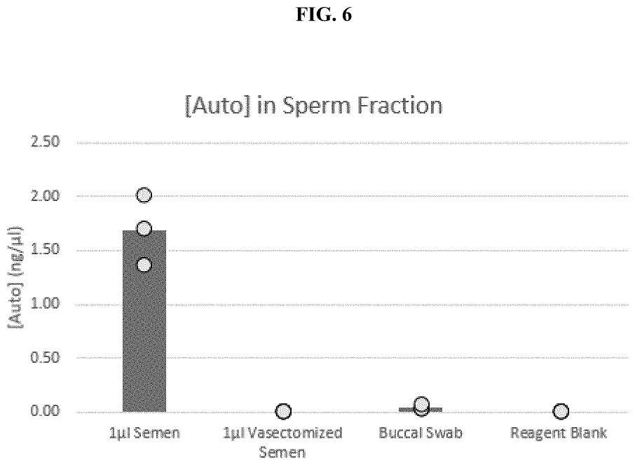

FIGS. 7A-7D show data from spotted buccal swabs. Data was obtained from a single experiment; each dot represents a sample (n=4 per condition) while bars and error bars represent the mean and standard deviation for each condition, respectively. Spotted buccal swabs were used as the input material for each sample. Data was quantified using Promega's POWERQUANT™ system.

FIGS. 8A-8D show data from post coital swabs. Data was obtained from a single experiment; each dot represents a sample (n=2 per condition) while bars and error bars represent the mean and standard deviation for each condition, respectively. Post coital vaginal swabs, collected 12 hours post coitus and processed 18 months after collection, were used as the input material for each sample. Data was quantified using Promega's POWERQUANT™ system.

### DEFINITIONS

To facilitate an understanding of the present technology, a number of terms and phrases are defined below. Additional definitions are set forth throughout the detailed description.

The terms “nucleic acid”, “polynucleotide”, “nucleotide sequence”, and “oligonucleotide” are used interchangeably herein and refer to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The terms encompass any deoxyribonucleotide, ribonucleotide, or peptide nucleic acid component, and any chemical variants thereof, such as methylated, hydroxymethylated, or glycosylated forms of these bases. The polymers or oligomers may be heterogenous or homogenous in composition, may be isolated from naturally occurring sources, or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. The terms “nucleic acid” and “nucleic acid sequence” may also encompass a chain comprising non-natural nucleotides, modified nucleotides, and/or non-nucleotide building blocks that can exhibit the same function as natural nucleotides (e.g., “nucleotide analogs”).

As used herein, the term “sperm-associated DNA” refers to any DNA sequence that is present within, or otherwise attached to, a sperm cell.

The terms “peptide”, “polypeptide”, and “protein” are used interchangeably herein and refer to a polymeric form of amino acids comprising at least two or more contiguous amino acids, which can include coded and non-coded amino acids, chemically or biochemically modified or derivatized amino acids, and polypeptides having modified peptide backbones.

The term “differential extraction” refers to extraction methods utilized to extract a subset of cell types from a heterogeneous population of cells. In certain embodiments, differential extraction includes the selective lysis of non-sperm cells in a mixture of sperm cells and non-sperm cells, including, but not limited to, epithelial cells. Differential extraction also is referred to in the art as “differential isolation.”

The term “biological sample”, as used herein, refers to any sample that contains at least one biological material. Exemplary biological materials include, but are not limited to, blood, saliva, skin, feces, urine, sperm cells, epithelial cells (including, but not limited to, vaginal epithelial cells), muscle tissue, and bone.

The term “forensic sample”, as used herein, refers to a biological sample obtained to address identity issues arising in legal contexts, including, but not limited to murder, rape, trauma, assault, battery, theft, burglary, other criminal matters, identity, parental or paternity testing, and mixed DNA samples.

The term “epithelial cell”, as used herein, refers to a cell that forms the tissues that line the outer surfaces of organs and blood vessels throughout an animal (e.g., a mammal) as well as the inner surfaces of cavities in many internal organs.

The term “sperm cell”, as used herein, refers to the reproductive cell of a male animal, such as a male mammal (e.g., a human), which can unite with an egg cell of a female animal to form a zygote.

The terms “digestion agent” and “lysis agent” are used interchangeably herein to refer to a substance or compound that disrupts and breaks open (lyses) cells. A digestion agent or lysis agent may be present in a buffer, which typically is referred to in the art as a “lysis buffer”. Examples of digestion or lysis agents include, but are not limited to, detergents (Tween-20, Triton X-100), surfactants (e.g., sodium dodecyl sulfate (SDS) or sodium lauryl sulfate (SLS)), and proteases (e.g., proteinase K). Certain exemplary lysis buffers are known in the art, and one skilled in the art can select a lysis buffer based on the intended use. In some embodiments, a concentration of a particular digestion agent exists in which the digestion agent is capable of disrupting and breaking open non-sperm cells (e.g., epithelial cells), but not sperm cells; at a higher concentration, the digestion agent will also disrupt and breaks open sperm cells.

The term “lysate” refers to a liquid phase containing lysed cell debris and DNA. A “whole cell lysate” contains all the contents of a lysed cell. A “processed cell lysate” has been processed (e.g., centrifuged, filtered, etc.) to remove a portion of the contents from the cell lysate. Certain embodiments herein utilize whole or processed cell lysates. Embodiments herein referring to “cell lysates” may find use with whole or processed cell lysates, unless otherwise indicated.

The term “reducing agent”, as used herein, refers to an agent that reduces disulfide bonds, e.g., in proteins, by donating an electron to another chemical species in a redox chemical reaction. In certain embodiments, a reducing agent disrupts protamine disulfide bridges in sperm cells and disulfide bonds that maintain the structure of the sperm head. Disulfide bond reducing agents can be water-insoluble or water soluble. Exemplary reducing agents include, but are not limited to, dithiothreitol (DTT), Tris(2-carboxyethyl)phosphine hydrochloride (TCEP), glutathione (GSH), 1-thioglycerol (1-TG), and mercaptoethanol (ME).

The term “cation exchange chromatography” refers to a type of ion exchange chromatography (IEX), which is used to separate molecules based on their net surface charge. More specifically, cation exchange chromatography uses a negatively charged ion exchange resin with an affinity for molecules having net positive surface charges. Cation exchange chromatography may be used both for preparative and analytical purposes and can separate a wide range of biomolecules, such as amino acids, nucleotides, and large proteins.

The term “cartridge” refers to a system that comprises a plurality of compartments and does not contain sufficient fluid and/or magnetic particle handling mechanisms to function independently of a separate fluid-handling and/or magnetic particle-handling instrument. A cartridge may be, in certain embodiments, designed for a single use, after which it is discarded. In certain embodiments, one or more of the compartments in a cartridge contains a reagent. In other embodiments, all of the compartments of a cartridge are contained in a single unit. In certain embodiments, the compartments of a cartridge are divided between two or more units that together form the cartridge. A single cartridge may be designed to process 1, 2, 4, 6, 8, 12, 16, 24, 48, 96, or more than 96 samples. In various embodiments, a cartridge is designed to process between 1 and 48 samples, between 1 and 24 samples, between 2 and 24 samples, between 1 and 16 samples, or between 2 and 16 samples. When a cartridge is designed to process at least two samples, it may be designed to process at least two of the samples simultaneously. In certain embodiments, a cartridge is designed to process all of the samples simultaneously.

### DETAILED DESCRIPTION

Provided herein are methods, reagents, and kits for the isolation of sperm cells from other cell types and the purification of sperm-associated DNA from a complex sample. In particular, methods, reagents, and kits are provided for the separation of partially (e.g., flagellum may be removed, but the sperm head is intact) or fully intact sperm cells from epithelial cells via binding of sperm cells to a cation exchange resin, and the extraction of sperm-associated DNA therefrom.

Experiments conducted during development of embodiments described herein demonstrate a direct binding interaction between sperm cells and a cation exchange resin (e.g., a paramagnetic cation exchange resin). Methods, reagents, and kits described herein utilize this interaction for the separation of sperm cells from sample contaminants and epithelial cells, and for the purification of sperm-associated DNA from the sample. In some embodiments, provided herein are processes for binding sperm cells, and their associated DNA, to a paramagnetic cationic exchange resin; this binding interaction provides for streamlined and automated methods for handling the sperm cells, isolating sperm-associated DNA, and for separating them from other contaminating substances, such as epithelial cells and contaminating DNA.

Methods described herein, and kits, reagents, and devices for performing such methods, differ from some existing methods in that sperm cells are separated partially or fully intact from a sample (e.g., containing non-sperm cells (e.g., epithelial cells), containing other contaminants (e.g., a sample collection device (e.g., swab tip), buffer, biological fluid components, environmental debris, etc.), and sperm-associated DNA is subsequently extracted/purified from the isolated sperm cells. In common differential extraction techniques, use is made of the differential lysis of non-sperm (e.g., epithelial cells) and sperm cells to isolate DNA from each cell type. These methods rely on the laborious physical separation (e.g., repeated centrifugation steps) of sperm cells from non-sperm cell lysates to allow isolation of sperm-associated DNA that is free of non-sperm (e.g., epithelial cells) DNA contamination. The methods and reagents provided herein allow for simplification of this process by binding sperm cells to paramagnetic particles allowing automation of the process of sperm cell enrichment and sperm-associated DNA purification. The disclosure provides a method comprising (a) contacting a sample comprising sperm cells with a digestion agent capable of efficiently lysing epithelial cells but not sperm cells; (b) separating a sperm-cell-enriched fraction of the sample from a sperm-cell-depleted fraction of the sample; (c) contacting the sperm-cell-enriched fraction with a cationic exchange resin; (e) washing the cationic exchange resin with a wash buffer; and (f) eluting sperm-associated DNA from the cationic exchange resin. The sample may be any suitable biological sample described herein. In some embodiments, the sample is a forensic sample which may comprise a mixture of different cell types, depending on the source of the sample. For example, the sample may comprise a mixture of epithelial cells and sperm cells.

A sample comprising sperm cells may be contacted (e.g., mixed) with any suitable digestion agent that is capable of efficiently lysing epithelial cells but not sperm cells. In some embodiments, a sample is contacted with a suitable concentration of a digestion agent that is capable of efficiently lysing epithelial cells but not sperm cells. In some embodiments, at a higher concentration, the same digestion agent is also capable of lysing sperm cells. It will be appreciated that sperm cells are protected from conventional digestion agents and lysis buffers due to the high abundance of disulfide bonds on sperm cells. Any suitable digestion agent known in the art and described herein may be used. In some embodiments, the digestion agent comprises a protease, such as proteinase K. In other embodiments, the digestion agent comprises a surfactant. Ideally, the digestion agent comprises both a protease and a surfactant. For example, the digestion agent may comprise proteinase K and sodium dodecyl sulfate (SDS). Whatever digestion agent is employed, the digestion agent desirably lyses at least 95% (e.g., 96%, 97%, 98%, 99%, or 100%) of the epithelial cells present in the sample. Along the same line, the digestion agent desirably lyses less than 25% (e.g., 20%, 15%, 10%, 5%, or 0%) of sperm cells present in the sample. In some embodiments, the digestion agent comprises a starting or final concentration of 0.01% to 1.0% (e.g., 0.01%, 0.02%, 0.05%, 0.1%, 0.2%, 0.5%, 1.0%, or ranges therebetween) surfactant (e.g., SDS). In some embodiments, the digestion agent comprises a starting or final concentration of 1 μg/ml to 100 μg/ml (e.g., 1 μg/ml, 2 μg/ml, 5 μg/ml, 10 μg/ml, 50 μg/ml, 100 μg/ml, or ranges therebetween) protease (e.g., proteinase K).

Due to the anionic nature SDS, excess amounts of SDS may inhibit the association between sperm cells and the cation exchange resin during the binding reaction. Thus, in some embodiments, the disclosed method may comprise diluting the SDS with a buffer (e.g., a Tween-containing buffer such as 0.5% Tween 20) for binding to the cationic exchange resin. For example, a 10-fold dilution has been shown to be sufficient to restore the association between sperm cells and the cation exchange resin. Any suitable dilution of SDS may be employed, however.

In some embodiments, the digestion agent further comprises a blocking agent. The term “blocking agent,” as used herein, refers to a compound that reduces non-specific binding of non-sperm DNA (e.g., epithelial cell DNA) to a cationic exchange resin. More specifically, in some embodiments, a blocking agent is a compound that mimics properties of human genomic DNA and competes against non-sperm DNA for potential binding sites on the cationic exchange resin than may allow non-sperm DNA to carry over into a sperm-cell-enriched fraction. Any suitable blocking agent may be included in the digestion agent, such as, for example, Herring Sperm DNA, poly-deoxy-inosinic-deoxy-cytidylic acid (poly d(I-C)), or bovine serum albumin (BSA).

In other embodiments, other additives or compounds may be used to selectively disrupt non-sperm DNA-binding interactions without significantly disrupting binding of the sperm DNA to the cationic exchange resin. Such additives may include, but are not limited to, molecules that selectively and tightly interact with DNA, such as polyethylenimine (PEI) and spermine.

In some embodiments, once sufficient digestion of epithelial cells (but not sperm cells) present in the sample has occurred, removal or inactivation of the digestion agent provides for optimal recovery of sperm-associated DNA. In this regard, the digestion agent may be removed from the sperm cells or inactivated, either actively or passively (e.g., by allowing the digestion agent to become inactivated). When the digestion agent comprises proteinase K, inactivation desirably comprises providing sufficient time to allow the proteinase K to self-inactivate (i.e., auto-proteolyze). Ideally, autoproteolysis of proteinase K is allowed to proceed to at least 95% (e.g., 96%, 97%, 98%, 99%, or 100%) inactivation of the proteinase K. In some embodiments, autoproteolytic digestion of the proteinase K is accelerated by depletion of calcium in the reaction mixture. Other methods for inactivating digestive agents include, for example, heat treatment (e.g., incubation above 70° C.), a change in pH, or the use of a chemical inhibitor.

In certain embodiments, the digested sample may be centrifuged to remove any solid substrate present in the initial sample (e.g., a swab) and pellet the partially or fully intact sperm cells. The resulting supernatant may be retained as a sperm-cell-depleted fraction. In other words, the method may comprise separating a sperm-cell-enriched fraction (i.e., centrifuged pellet) of the sample from a sperm-cell-depleted fraction (i.e., supernatant) of the sample. It will be appreciated that the sperm-cell-depleted fraction contains epithelial cells present in the sample (e.g., lysed epithelial cells), and the sperm-cell-depleted fraction may be retained for subsequent analysis or discarded. It will be appreciated that complete removal of the supernatant containing the sperm-cell-depleted fraction will result in optimal purity of the sperm-cell-enriched fraction; however, in some embodiments, some supernatant (e.g., up to about 50 μl) may remain with the pellet.

In some embodiments, sperm-depleted DNA may be obtained from the sperm-cell-depleted fraction and analyzed. For example, sperm-depleted DNA may be quantified or genotyped. Methods for quantifying DNA are known in the art and include, for example, spectrophotometry, fluorescence, real-time PCR (also known as quantitative PCR (qPCR)), and digital PCR technologies. Genotyping the sperm-depleted DNA may be performed using any suitable method known in the art. Such methods include, for example, short tandem repeat (STR) analysis, restriction fragment length polymorphism (RFLP) of genomic DNA, random amplified polymorphic detection (RAPD) of genomic DNA, amplified fragment length polymorphism (AFLP) detection, single nucleotide polymorphism (SNP) detection, polymerase chain reaction (PCR), DNA sequencing, allele specific oligonucleotide (ASO) probes, and hybridization to DNA microarrays or beads.

In certain embodiments, the sperm-depleted DNA is genotyped using short tandem repeat (STR) analysis. STRs are repetitive sequence elements 3-7 base pairs (bp) in length scattered throughout the human genome. By amplifying and analyzing these polymorphic loci, and then comparing the resulting STR profile to that of a reference sample, the origin of biological samples such as cells or tissues can be identified and verified. STR analysis is used in forensic science to evaluate specific STR regions found on nuclear DNA. In various embodiments, loci containing 3 bp (trinucleotide), 4 bp (tetranucleotide), and/or 5 bp repeat sequences are used for human identification. Four and 5 bp repeat sequences are found throughout the human genome and are, in certain instances, highly polymorphic. The number of alleles at a tetranucleotide repeat STR locus ranges, in various embodiments, from about 4 to about 50.

In certain embodiments, when isolated DNA is used for detection of polymorphic STRs, the amplified alleles from the individual DNA samples can be compared to one or more size standards, e.g., commercial DNA markers and/or locus-specific allelic ladders, to determine the alleles present at each locus. In certain embodiments, allelic ladders comprise two or more distinct lengths of DNA representing two or more known alleles from a particular locus. DNA may be visualized by any suitable technique known in the art, including, but not limited to, silver staining, radioactive labeling, fluorescent labeling, and using various dyes and stains. In certain embodiments, prior to visualization, DNA is separated using denaturing or native gel electrophoresis, or any other size separation method. In certain embodiments, amplified alleles are subjected to DNA sequence analysis. Exemplary methods for DNA amplification, genotyping, and analysis are known in the art and are described, e.g., Butler, John M., Advanced Topics in Forensic DNA Typing: Methodology, Elsevier Inc.: MA, USA (2011).

The disclosed method may further comprise resuspending the pellet remaining from centrifugation (containing the sperm cell-enriched fraction), along with any residual supernatant (containing the sperm cell-depleted fraction), in a binding buffer (e.g., Tris-HCl (100 mM, pH 7.3) and Tween 20 (0.5%) or SDS (0.1%)). The binding buffer may further comprise a blocking agent, as described above.

The sperm-cell-enriched fraction may be contacted with any suitable cationic exchange resin, and sperm cells can be isolated from the sample by virtue of their ability to bind a cationic exchange resin with high affinity. A variety of cationic exchange resins and matrices that may be used in the disclosed methods and kits are known in the art and are commercially available (see, e.g., Ion Exchange Chromatography, Principles and Methods, GE Healthcare Handbook 11000421; and Weiss, J. (ed.), Handbook of Ion Chromatography, 4^{th} ed, Wiley-VCH (2016)). In some embodiments, the cationic exchange resin comprises particles that contain an iron core or an iron oxide core. In certain embodiments, the cationic exchange resin comprises a paramagnetic cellulose resin. In certain embodiments, the cationic exchange resin comprises a silica- and/or polymer-based paramagnetic resin. The term “paramagnetic,” as used herein, refers to a body or substance that, when placed in a magnetic field, possesses magnetization in direct proportion to the field strength.

A paramagnetic cationic exchange resin as described herein may be used with the MAXWELL® RSC 48 Instrument and MAXWELL® 16 Instrument (Promega, Madison, Wis.). The MAXWELL® RSC 48 Instrument is a compact, automated nucleic acid purification platform that processes up to 48 samples simultaneously. MAXWELL® 16 instruments purify samples using paramagnetic particles (PMPs), which provide a mobile solid phase that optimizes capture, washing, and elution of the target material. MAXWELL® 16 instruments are magnetic particle handlers that efficiently preprocess liquid and solid samples, transport the PMPs through purification reagents in the prefilled cartridges, and mix during processing (see Technical Manuals for MAXWELL® 16 (TM295), MAXWELL® 16 Forensic (TM321), MAXWELL® FSC (TM462), and MAXWELL® RSC 48 (TM510) instruments).

The cationic exchange resin and the sperm cell-containing sample desirably are incubated for a sufficient amount of time to ensure efficient binding of sperm cells to the resin. For example, the sperm cell-containing sample and the cationic exchange resin may be incubated together for at least about 5 minutes, about 10 minutes, about 15 minutes, about 20 minutes, about 30 minutes, about 45 minutes, about 50 minutes, about 1 hour, about 90 minutes, or about 2 hours. Following a suitable incubation time, the cationic exchange resin is washed with a wash buffer. The term “wash buffer,” as used herein, refers to a buffer which removes contaminants from the cationic exchange resin, while allowing sperm cells to remain bound to the cationic exchange resin. Exemplary wash buffers include, a detergent (e.g., SDS, Tween 20), pH buffer (e.g., phosphate buffered saline (PBS), Tris-HCl, pH 7.3, etc.), salt (e.g., NaCl, KCl, MgCl₂ etc.), and/or other common buffer components. One of ordinary skill in the art can select a suitable wash buffer according to the sample type and reaction conditions.

In certain embodiments, the sperm-associated DNA may be eluted from the cationic exchange resin using an elution buffer. The term “elution buffer,” as used herein, refers to a buffer that releases sperm-associated DNA, in particular by lysing the sperm cells bound to the sperm-binding particles. Certain exemplary elution buffers may comprise a detergent (as described herein), a digestion agent (as described herein), and/or a reducing agent (as described herein). In certain embodiments, the elution buffer comprises a detergent, a digestion agent and, a reducing agent. For example, the detergent may comprise SDS and/or Tween 20, the digestion agent may comprise proteinase K, and the reducing agent may comprise DTT, TCEP, βME, and/or 1-TG. One of ordinary skill in the art can select a suitable elution buffer according to the sample type and cationic exchange resin used. In certain embodiments, the sperm-associated DNA may be eluted from the cationic exchange resin by heating the cationic exchange resin in the elution buffer. For example, the cationic exchange resin may be heated by raising the temperature to at least 65° C.

In some embodiments, a reducing agent may be added in a cell lysis and/or elution step. Exemplary reducing agents that can be employed in the disclosed method are known in the art. Such reducing agents include, but are not limited to, dithiothreitol (DTT), tris(2-carboxyethyl)phosphine (TCEP), 2-mercaptoethanol (βME), and/or 1-thioglycerol (1-TG). When the reducing agent is 1-TG, the concentration of 1-TG added to the sperm cells is between about 1 mM and about 25 mM (e.g., 1 mM, 2 mM, 3 mM, 4 mM, 5 mM, 6 mM, 7 mM, 8 mM, 9 mM, 100 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, or ranges therebetween). When other reducing agents are used, concentrations sufficient to yield similar reduction of disulfide bonds as the 1-TG concentrations above and used herein may be employed.

The sperm-associated DNA eluted from the cationic exchange resin may then be analyzed. For example, the sperm-associated DNA may be quantified or genotyped, using similar methods as previously described herein with respect to analysis of a sperm cell-depleted fraction. In certain embodiments, the eluted sperm-associated DNA is genotyped using STR analysis.

The disclosure further provides a method of forensic analysis which comprises performing any of the method steps described herein, and comparing the sperm-associated DNA of the sample with a reference sample and/or a database. The term “forensic analysis” is used generally to refer to an investigation of a crime or security incident. The analysis of DNA samples obtained from a suspect or victim, often referred to in the art as “DNA profiling,” is a major component of forensic analyses, depending on the nature of the crime or security incident. The term “reference sample,” as used herein, refers to a sample of an individual's DNA (e.g., a crime suspect or victim) against which the sperm-associated DNA is compared. Reference samples are typically collected through a buccal swab. The reference sample is analyzed, e.g., by genotyping methods described herein, to create the individual's DNA profile. The DNA profile is then compared against the sperm-associated DNA isolated in accordance with the methods disclosed herein to determine whether there is a genetic match. In other words, the method comprises determining whether a particular subject is the source of the sperm cells in the sample. In some embodiments, the sperm-associated DNA may be compared against DNA sequences in a database or other repository. Exemplary databases include, but are not limited to, the Combined DNA Index System (CODIS), which is maintained by the U.S. Federal Bureau of Investigation (FBI), and the United Kingdom National DNA Database (NDNAD).

In certain embodiments, one or more of the method steps described herein may be automated. It will be appreciated that automated sample preparation provides significant benefits to laboratories, allowing users to focus on more detailed work while reducing the risk of exposure to chemicals and the need for repetitive motions, such as pipetting. In this regard, one or more of the steps described herein may be performed by an automated liquid handler. Automated liquid handlers allow laboratories to handle increased sample processing demands while minimizing the need to increase headcount. Automated liquid handlers are commercially available from a variety of sources, including, but not limited to, the MAXPREP™ Liquid Handler combined with the MAXWELL® extraction available from Promega Corp. (Madison, Wis.).

In addition to or alternatively, one or more of the steps described herein may be performed by an automated particle handler, which moves particles (e.g., paramagnetic particles) instead of liquids. Automated particle handlers present minimal risk of cross-contamination because no liquid handling or splashing occurs during sample processing.

A general exemplary embodiment of the methods described herein comprises (1) pre-processing steps and (2) purification steps. The exemplary pre-processing steps begin by digesting a sample and/or substrate in a digestion buffer containing detergent (e.g., sodium dodecyl sulfate (SDS), proteinase K (ProK), and buffer (e.g., Tris.HCl). The sample is incubated in the digestion buffer at elevated temperature (e.g., 50-90° C.). The digestion step lyses epithelial cells while leaving sperm cells partially intact (e.g., flagellum may be removed, but the sperm head is intact); sperm cells are protected from lysis due to their high abundance of di-sulfide bonds. Following the initial digestion, samples are transferred to a device configured to allow flow through of liquid but to retain a solid support/substrate (e.g., swab). Centrifugation of the device results in the solid support/substrate (e.g., swab) being retained in an upper chamber, flow-through of the sample and buffer into a collection tube, and pelleting of the sperm cells at the bottom of the collection tube. The centrifugation step facilitates (1) removal of a solid sample substrate (such as a swab, if present) and (2) pelleting the sperm cells to the bottom of the centrifuge tube. Following the centrifugation step, all or some of the supernatant is removed; which can be saved and later analyzed as the non-sperm fraction as it contains the DNA from any epithelial cells that may be present in the sample. The pellet fraction, possibly along with residual supernatant fraction, is resuspended in a binding buffer and optionally a surfactant (e.g., Tween 20). The exemplary purification steps begin by combining the resuspended pellet fraction with a paramagnetic cationic exchange resin. Mixing of the resuspended pellet fraction with a paramagnetic cationic exchange resin allows for binding between the sperm cells and the cationic exchange resin. The bound sperm cells are washed (e.g., multiple washes times) in a wash buffer (e.g., Tris-HCl, pH 7.3 and Tween 20), and then the sperm cells are lysed via heating (e.g., 60-70° C.) for an extended time period (e.g., 10-60 minutes) with mixing in elution buffer (e.g., Tris-HCl (pH 8), 1-TG, SDS, and ProK) releasing the sperm-associated DNA. Upon elution from the resin, the elution buffer contains the purified sperm fraction.

A specific exemplary embodiment of the methods described herein begin by digesting a sample and/or substrate in a digestion buffer that contains 0.1% SDS, 1 mg/ml ProK, and 50 mM Tris.HCl (pH 8.0). 400 μl is a typical volume for use with standard swabs, a greater or lesser volume may be used as appropriate to fully submerge the sample and/or substrate. The sample is incubated in the digestion buffer at 70° C. for 50 minutes. The digestion step lyses epithelial cells while leaving sperm cells partially intact (e.g., flagellum may be removed, but the sperm head is intact); sperm cells are protected from lysis due to their high abundance of di-sulfide bonds. Following the initial digestion, samples are transferred to a Spin Basket (Promega; Madison, Wis.) placed inside a ClickFit tube (Promega; Madison, Wis.). Centrifugation of the Spin Basket/ClickFit system for 5 minutes at maximum speed (according to product specifications) results in the solid support/substrate (e.g., swab) being retained in an upper chamber, flow-through of the sample and buffer into a collection tube, and pelleting of the sperm cells at the bottom of the collection tube. The centrifugation step facilitates (1) removal of a solid sample substrate (such as a swab, if present) and (2) pelleting the sperm cells to the bottom of the centrifuge tube. Following the centrifugation step, the supernatant is removed by pipetting and the sperm cell pellet is retained in the tube. Up to 50 μl of liquid may be left behind in the tube, along with the pellet. The supernatant contains the non-sperm fraction and can be analyzed at the epithelial fraction (i.e., as it contains the DNA from any epithelial cells that may be present in the sample). The pellet fraction, along with any residual supernatant fraction, is resuspended in a 900 μL of binding buffer (100 mM Tris.HCl (pH 7.3) and 0.5% Tween 20). The resuspended pellet is transferred to Well 1 of a Maxwell® Cartridge. The other wells of the Maxwell® Cartridge contain: Well 2—paramagnetic cationic exchange resin (5 mg in 300 μL total volume (10% EtOH); Well 3-500 μL wash buffer (100 mM Tris-HCl (pH 7.3) and 0.5% Tween 20); Well 4—500 μL salt wash buffer (100 mM Tris-HCl (pH 7.3), 0.5% Tween 20, and 250 mM NaCl); Wells 5-7 (each)—500 μL wash buffer (100 mM Tris-HCl (pH 7.3) and 0.5% Tween 20); and Well 8—elution plunger. The paramagnetic cationic exchange resin from Well 2 is transferred to the wash buffer of Well 3 and quickly rinsed. The paramagnetic cationic exchange resin is then collected from Well 3 and transferred to Well 1 and mixed with the resuspended sperm cell pellet. Mixing of the resuspended pellet fraction with the paramagnetic cationic exchange resin allows for binding of the sperm cells to the cationic exchange resin. Binding is allowed to occur for 15 minutes, with periodic mixing. The bound sperm cells are transferred to Well 4 and washed in the salt wash buffer with constant mixing for about 140 seconds. The resin is subsequently transferred and washed (˜140 seconds/wash) in Wells 5-7 in wash buffer. The resin is then transferred to Well 8, and the sperm fraction is eluted from the resin into an elution buffer (50 mM Tris-HCl (pH 8), 100 μg/ml Proteinase K, 5.75 mM 1-TG, 0.1% SDS, Total Volume≤200 μl) at 65° C. for 30 minutes. Upon elution form the resin, the elution buffer contains the purified sperm fraction.

The above protocols are exemplary and are not limiting on the full scope of embodiments herein. Alternative reagents, buffers, concentrations, devices, order and number of steps, etc. are contemplated, as described herein.

The final eluate produced by the exemplary steps above contains the sperm-associated DNA and is compatible with quantification chemistries (e.g., POWERQUANT® system (Promega Corp.) and STR genotyping chemistries (e.g., POWERPLEX®16 HS system (Promega Corp.), POWERPLEX® ESI/ESX system (Promega Corp.), POWERPLEX® FUSION system, POWERPLEX® FUSION 6C system (Promega Corp.), IDENTIFILER® system (ThermoFisher Scientific), GLOBALFILER™ 6-DYE™ system (ThermoFisher Scientific), and INVESTIGATOR® 24Plex system (Qiagen)). Other embodiments of isolating/purifying sperm-associated DNA are contemplated and within the scope of the present disclosure.

The disclosure also provides a kit comprising two or more of: (a) a digestion agent capable of efficiently lysing epithelial cells but not sperm cells; (b) a cationic exchange resin; (c) a binding buffer; (d) a wash buffer; and (e) an elution buffer comprising a detergent, a digestion agent, and a reducing agent. Descriptions of the digestion agent, cationic exchange resin, wash buffer, elution buffer, and components thereof set forth above also apply to those same aspects of the aforementioned kit. The binding buffer may be any buffer suitable for putting pelleted cells and/or DNA back into suspension (resuspending) in a fluid and facilitating binding of a sample to the cation exchange resin. Suitable binding buffers are known in the art. An exemplary binding buffer comprises Tris-HCl (100 mM, pH 7.3) and Tween 20 (0.5%) buffer.

The kit also desirably comprises instructions for use. Instructions included in the kit may be affixed to packaging material or may be included as a package insert. The instructions may be written or printed materials but are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The kit may include a cartridge that comprises the cationic exchange resin, wash buffer, and one or more reagents useful for practicing the methods disclosed above. The cartridge may be disposable. The cartridge may include one or more containers holding the reagents, as one or more separate compositions, or, optionally, as admixture where the compatibility of the reagents will allow. The cartridge may also include other material(s) that may be desirable from a user standpoint, such as buffer(s), a diluent(s), and/or any other material useful in sample processing, washing, or conducting any other step of the method.

The various components of the kit optionally are provided in suitable containers as necessary.

The following example further illustrates the invention but, of course, should not be construed as in any way limiting its scope.

### Example

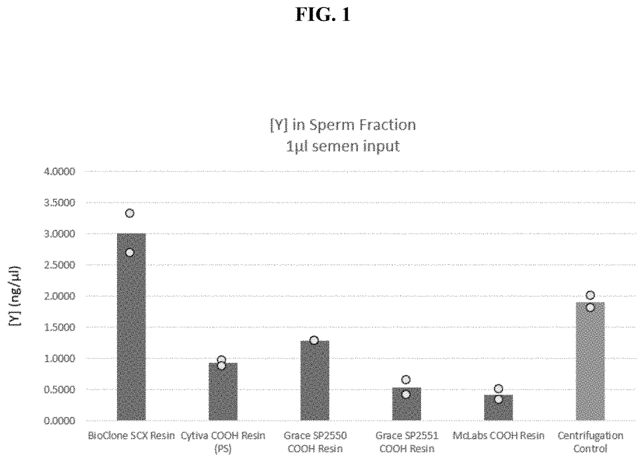

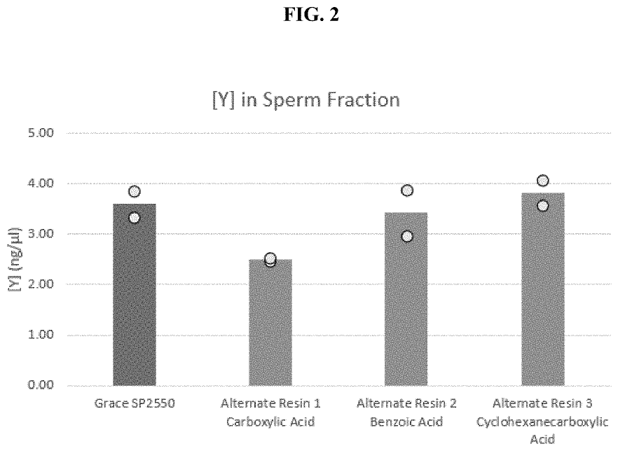

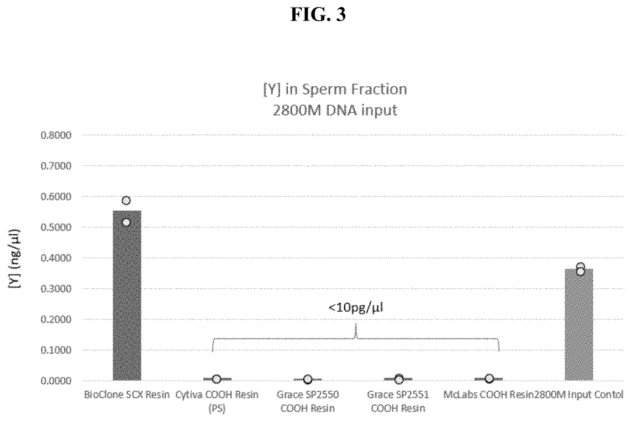

Initial pilot studies examined a variety of sulfonated and carboxylated resins to determine whether they could be used to selectively bind sperm cells. Many resins appeared to bind sperm cells to some degree, as shown in FIG. 1. Experiments demonstrated that several alternative cation exchange particles were capable of purifying sperm-associate DNA from semen samples (FIG. 2). This sperm binding appeared to have some degree of specificity as most of the resins also had very little direct DNA binding activity, with one exception (see FIG. 3).

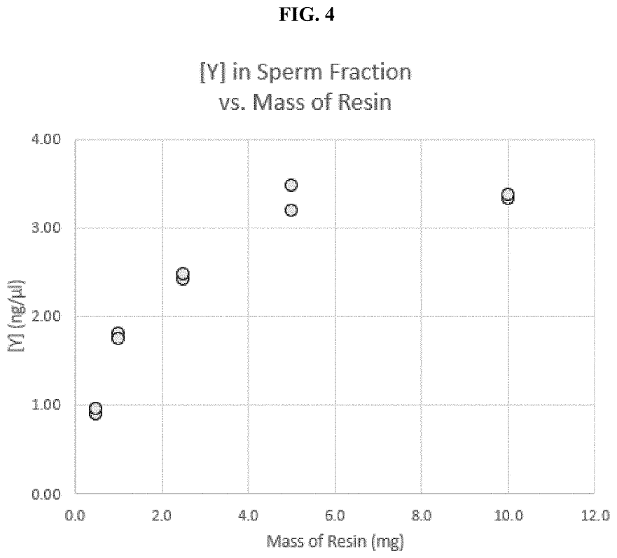

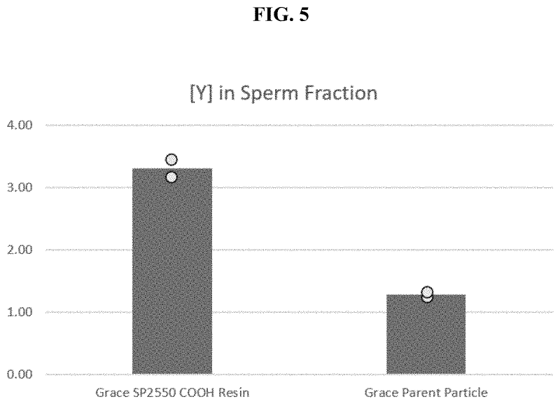

The Grace SP2550 particle was selected for further experimentation based on its low nonspecific DNA binding and its reasonably high sperm affinity. To confirm that the sperm cells were specifically interacting with the resin, the relationship between the mass of resin used in the Maxwell® cartridge and the yield of sperm-associated DNA ([Y]) in the sperm fraction was investigated (see FIG. 4). There was a clear dose-dependent relationship, which suggests that the resin is an integral part of the sperm binding mechanism, likely via the direct capture of sperm cells onto the resin. Next, the functionalized Grace SP2550 particle was compared to the non-functionalized parent particle (also by Grace) to determine whether the chemical groups responsible for the cationic exchange (e.g., COOH groups) were required for the capture of sperm cells. Though the parent resin captured sperm cells to some amount, the degree of capture was greatly enhanced by the addition of the COOH groups onto the surface of the particle (see FIG. 5).

To examine the specificity of the binding reaction, both vasectomized semen and buccal swabs (lacking any semen) were used as input materials for the prototype protocol. A reagent blank was also run in parallel to detect any potential contamination. For these experiments, ˜50 μl of epithelial supernatant was left in the tube with the pellet fraction, which should represent the conditions most likely to generate the highest degree of carryover. While an abundance of DNA was purified from the liquid semen sample (indicated by the [Auto] in the sperm fraction), no DNA was detected in the reagent blanks or the vasectomized semen samples and very little DNA (˜40 μg/μ1) was carried over into the sperm fraction from buccal swab samples.

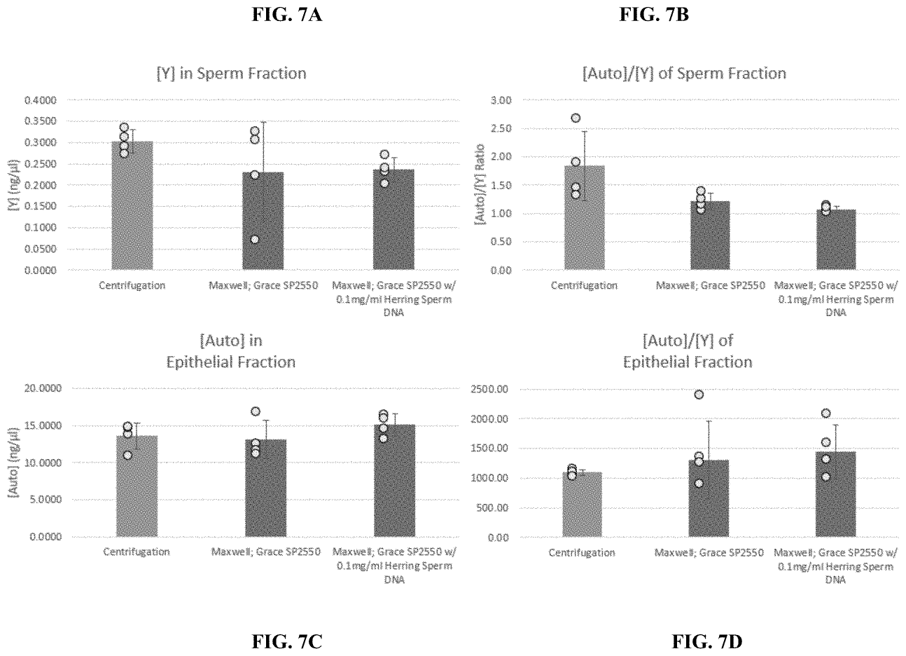

Next, this prototype protocol was tested with buccal swabs that had been spotted with 0.1 μl semen and allowed to air dry; these mock samples are intended to approximate sexual assault casework samples. Using these mock samples, the prototype differential extraction method was able to purify sperm-associated DNA equally as well as paired centrifugation method controls—yields, indicated by the [Y] in the sperm fraction, were ˜80% of control values while the purity, indicated by the [Auto]/[Y] ratio of the sperm fraction, was better for the prototype method. Notably, the prototype method extracted ˜40 ng of nearly pure male DNA from mock samples that contained over 6 μg of female DNA (see FIGS. 7A-7D). Additionally, inclusion of a blocking agent (Herring Sperm DNA, Promega Catalogue #: D1816, 0.1 mg/ml in the digestion buffer) further increased the purity of the sperm fraction without sacrificing yields.

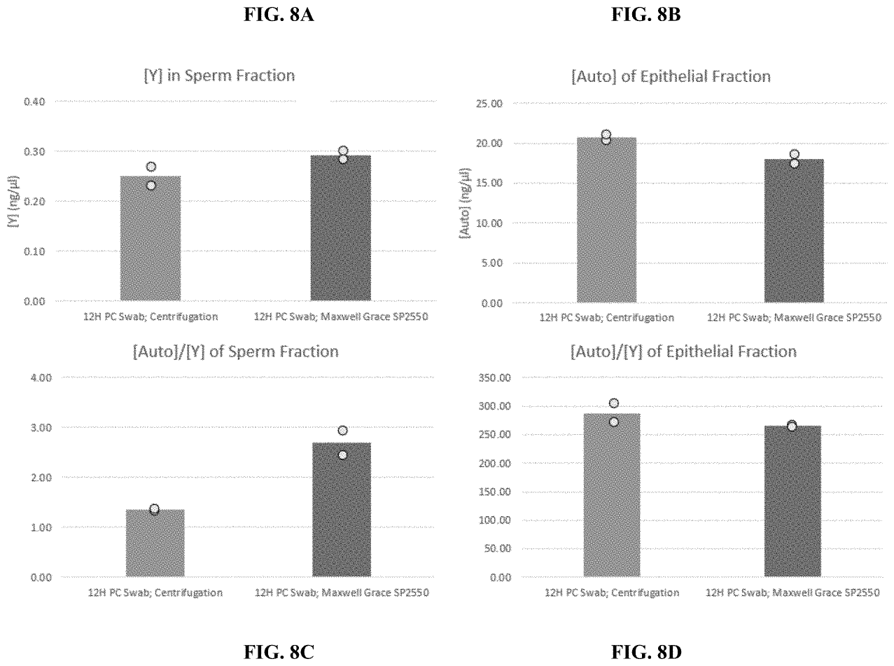

This prototype protocol was evaluated for separating sperm-associated DNA from post coital swabs. As shown in FIGS. 8A-8D, the prototype differential extraction method efficiently and effectively separated the sperm-associated DNA into the sperm fraction.

### REFERENCES

The following references are herein incorporated herein in their entireties.

Gill et al., “Forensic application of DNA ‘fingerprints,’” Nature, 318: 577-579 (1985);

Yoshida et al., “The modified method of two-step differential extraction of sperm and vaginal epithelial cell DNA from vaginal fluid mixed with semen,” Forensic Science International, 72: 25-33 (1995);

Vandewoestyne M and Deforce D., “Laser capture microdissection in forensic research: a review,” Int J Legal Med, 124: 513-521 (2010);

Garvin et al., “DNA Preparation from Sexual Assault Cases by Selective Degradation of Contaminating DNA from the Victim,” Journal of Forensic Sciences, 54: 1298-1303 (2009);

Anslinger et al, “Application of sperm-specific antibodies for the separation of sperm from cell mixtures,” Forensic Science International: Genetics Supplement Series, 1: 394-395 (2008);

Zhao et al., “Isolating Sperm from Cell Mixtures Using Magnetics Beads Coupled with an Anti-PH-20 Antibody for Forensic DNA Analysis,” PLOS ONE, 11: e0159401 (2016);

1. The method of claim 2, comprising a step between steps (a) and (b) of
separating a sperm-cell-enriched fraction of the sample from a sperm-cell-depleted fraction of the sample; wherein contacting the sample with a cationic exchange resin comprises
contacting the sperm-cell-enriched fraction with a cationic exchange resin.

2. A method comprising:
(a) contacting a sample comprising sperm cells with a digestion agent capable of efficiently lysing epithelial cells but not sperm cells;
(b) contacting the sample with a cationic exchange resin;
(c) washing the cationic exchange resin with a wash buffer; and
(d) eluting sperm-associated DNA from the cationic exchange resin

3. The method of claim 2, wherein the digestion agent comprises proteinase K.

4. The method of claim 2, wherein the digestion agent comprises sodium dodecyl sulfate (SDS).

5. The method of claim 2, wherein step (a) further comprises contacting the sample with a blocking agent.

6. The method of claim 5, wherein the blocking agent comprises Herring Sperm DNA or poly-deoxy-inosinic-deoxy-cytidylic acid (poly d(I-C)).

7. The method of claim 2, wherein the digestion agent is incubated with the sample at a temperature of at least about 37° C.

8. The method of claim 2, wherein at least 95% of epithelial cells present in the sample are lysed.

9. The method of claim 1, wherein the sperm-cell-depleted fraction is discarded.

10. The method of claim 1, wherein the sperm-cell-depleted fraction is retained for subsequent analysis.

11. The method of claim 10, further comprising obtaining sperm-depleted DNA from the sperm-cell-depleted fraction.

12. The method of claim 11, further comprising quantifying the sperm-depleted DNA.

13. The method of claim 11, further comprising genotyping the sperm-depleted DNA.

14. The method of claim 13, wherein genotyping the sperm-depleted DNA comprises performing short tandem repeat (STR) analysis.

15. The method of claim 1, further comprising a step of separating the sperm-cell-enriched fraction from the sperm-cell-depleted fraction.

16. The method of claim 15, wherein the sperm-cell-enriched fraction is separated from the sperm-cell-depleted fraction by centrifugation.

17. The method of claim 2, wherein the cation exchange resin is paramagnetic.

18. The method of claim 2, wherein the sperm cells bind to the cationic exchange resin.

19. (canceled)

20. The method of claim 2, wherein the sperm-associated DNA is eluted from the cationic exchange resin in an elution buffer comprising a detergent, a digestion agent, and a reducing agent.

21. (canceled)

22. The method of claim 20, wherein the detergent comprises SDS, the digestion agent comprises proteinase K and the reducing agent comprises DTT, TCEP, βME, GSH, and/or 1-TG.

23. The method of claim 20, wherein the sperm-associated DNA is eluted from the cation-exchange resin by heating the cationic exchange resin in the elution buffer to at least 65° C.

24. (canceled)

25. A method of conducting forensic analysis comprising performing the steps of claim 2, and comparing the sperm-associated DNA of the sample with a reference sample and/or a database.

26. The method of claim 25, further comprising determining whether a subject is the source of the sperm cells in the sample.

27. The method of claim 2, wherein one or more of the steps are automated.

28-29. (canceled)

30. The method of claim 25 further comprising comparing the sperm-depleted DNA of the sample with a reference sample and/or a database.

31. The method of claim 30, further comprising determining whether a subject is the source of the sperm-depleted DNA in the sample.

32. A kit comprising two or more of:
(a) a digestion agent capable of efficiently lysing epithelial cells but not sperm cells;
(b) a cationic exchange resin;
(c) a binding buffer;
(d) a wash buffer; and
(e) an elution buffer comprising a digestion agent and a reducing agent.

33-36. (canceled)
